(19) United States
(12) Patent Application Publication
(10) Pub. No.: US 2026/0114877 A1
(43) Pub. Date: Apr. 30, 2026

(54) Tourniquet

(71) Applicant: Steven B. King, Las Cruces, NM (US)

(72) Inventor: Steven B. King, Las Cruces, NM (US)

(21) Appl. No.: 18/831,402

(22) Filed: Jan. 10, 2025

Related U.S. Application Data
(60) Provisional application No. 63/630,385, filed on Jan. 26, 2024.

(51) Int. Cl. A61B 17/132; A61B 5/024; A61B 17/00

(52) U.S. Cl. CPC A61B 17/132; A61B 5/024; A61B 2017/00199; A61B 2017/00407

(57) ABSTRACT

An electronically controlled tourniquet device.

### BACKGROUND OF THE INVENTION

### 1. Field of the Invention

This invention relates to a tourniquet device which utilizes electronic monitoring to prevent injury.

### 2. Description of the Prior Art

The use of tourniquet devices to cut down on pain and hemorrhaging to an injured limb has been around since the Middle Ages. A band is placed around the injured limb and tightened using any suitable tightening device until blood flow from the injury is curtailed. Some tourniquet designs use a ratchet mechanism that must be continuously tightened to remove excess slack. Some tourniquets use an “H” clip that requires pulling the tourniquet to the desired tightness and any excess material is wrapped around the “H” clip to secure the tourniquet in place. Most modern tourniquets use what is referred to as a windlass, which is a stick shaped object that you twist until the desired tightness is achieved.

It is the tightening of the tourniquet that has and can result in injury to the damaged limb. If the tourniquet is tightened too much, the limb could be damaged so much that it must be amputated. If the tourniquet is not tightened enough then blood flow will continue which could result in death. For such reasons, modern tourniquets require constant monitoring by the user so that the correct amount of pressure is applied. U.S. Pat. Nos. 5,048,536 and 5,931,853 issued to McEwen as well as U.S. Pat. Nos. 7,166,123 and 7,485,131 issued to Hovanes all require and operator to monitor and adjust the pressure applied by the tourniquet. For someone untrained in the use of tourniquets, the potential injury to the patient increases exponentially. For such reasons, a need exist to electronically monitor and properly tighten the tourniquet.

### SUMMARY OF THE INVENTION

It is, therefore, an object of the present invention to provide a tourniquet which is automatically controlled through the use of an electronic ratcheting device in combination with an integrated or external pulse monitor. Other objects and advantages of the present invention will become apparent from the following detailed description when viewed in conjunction with the accompanying drawings, which set forth certain embodiments of the invention.

### BRIEF DESCRIPTION OF THE DRAWINGS

FIGS. 1 and 2 are isometric drawings of the tourniquet of the present invention.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for the claims and as a basis for teaching one skilled in the art how to make and/or use the invention.

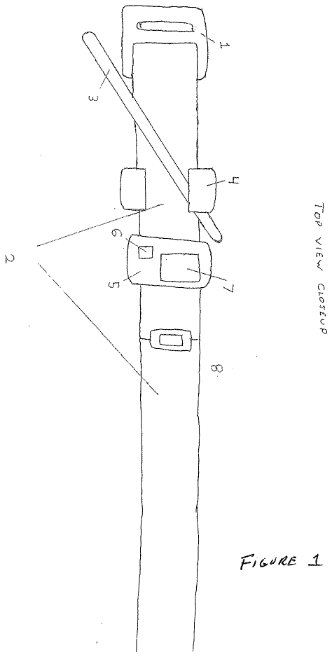

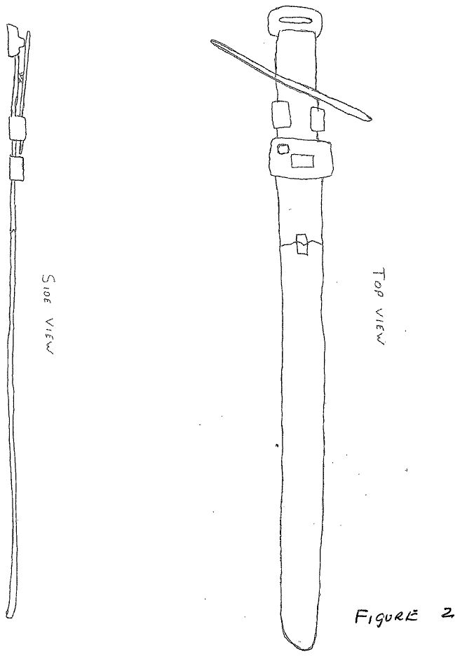

The device of the present invention, as depicted in FIGS. 1 and 2, is comprised of a belt [2] used to wrap the limb and provide tension around the wound for restricting blood flow. The belt [2] can be made of any common material rigid enough to wrap a person's limb but pliable enough to fit multiple limb dimensions. On one end of the belt [2] is a buckle [1] through which the other end of the belt [2] is fed to secure the tourniquet.

Shown in FIGS. 1 and 2 is a windlass rod [3] with windlass clip [4] for manually tightening the belt [2]. What sets apart the tourniquet of the present invention and those currently existing is the electronic ratcheting device [5]. Instead of tightening the belt [2] through use of the windlass rod [3] and windlass clip [4], the belt [2] is tightened automatically using the electronic ratcheting device [5], activated by pushing the start button [6]. A display screen [7] can be used to provide information to the user such as overall device tension, blood pressure and the date/time of application. Not shown in either FIG. 1 or 2 is the integrated or external pulse monitor which controls how much the electronic ratcheting device is tightened. In case or emergency situations or complications, a quick release [8] button is provided. Alternatively to the foregoing, a sensor can be attached to the tourniquet which will detect a pulse. If at any point, a pulse is detected, the sensor can communicate electronically with the electronic ratcheting device to re-engage until the pulse stops again. While not necessary for normal operation of the tourniquet, the tourniquet can be equipped with a quick release mechanism for emergency situations which will rapidly disengage the belt [2] from the buckle [1].

While the preferred embodiments have been shown and described, it will be understood that there is no intent to limit the invention by such disclosure, but rather, is intended to cover all modifications and alternate constructions falling within the spirit and scope of the invention as defined in the appended claims.

1. An tourniquet that includes an electronic ratcheting devices and an external or integrated pulse monitor which communicates with and controls the ratcheting device.

2. The tourniquet as in claim 1 wherein the electronic ratcheting device comprises a set of internal gears which will rotate to increase or decrease the slack in the tourniquet resulting in either increased or decreased pressure.

3. The tourniquet of claim 2 wherein the ratcheting device is preset with the medically recommended standard amount of pressure to cause occlusion.

4. The tourniquet of claim 2 further comprising a sensor to detect a pulse.

5. The tourniquet of claim 4 wherein if a pulse is detected by the sensor, the sensor will electronically communicate with the electronic ratcheting device will re-engage until a pulse is no longer detected.

6. The tourniquet of claim 1 wherein the tourniquet can be manually operated using the windlass rod and windlass clip should the electronic ratcheting device fail.

7. The tourniquet of claim 1 further comprising a start button which will initiate the electronic ratcheting device.

8. The tourniquet of claim 1 further comprising a display screen to display pertinent information such as date/time of application, pulse reading, battery level and any tourniquet failures.

9. The tourniquet of claim 1 further comprising a quick release mechanism.
